(19) United States
(12) Patent Application Publication
(10) Pub. No.: US 2018/0092949 A1
(43) Pub. Date: Apr. 5, 2018

(54) PREVENTION OR TREATMENT OF AN INFECTION BY BACTEROIDES THETAIOTAOMICRON

(71) Applicants: INSTITUT NATIONAL DE LA RECHERCHE AGRONOMIQUE, PARIS (FR); UNIVERSITE PIERRE ET MARIE CURIE (PARIS 6), PARIS (FR); ASSISTANCE PUBLIQUE - HÔPITAUX DE PARIS, PARIS (FR)

(72) Inventors: HARRY SOKOL, PARIS (FR); PHILIPPE LANGELLA, VELIZY-VILLACOUBLAY (FR); THOMAS WALTER HOFFMANN, LE MANS (FR); CHANTAL BRIDONNEAU, SACLAY (FR); HANG PHUONG PHAM, PARIS (FR); MATHIAS LAVIE-RICHARD, SAINT CYR L'ECOLE (FR)

(21) Appl. No.: 15/565,947

(22) PCT Filed: Apr. 22, 2016
(86) PCT No.: PCT/FR2016/050947

§ 371 Date: Oct. 12, 2017

(30) Foreign Application Priority Data
Apr. 23, 2015 (FR) 1553645

(51) Int. Cl. A61K 35/74; A23L 33/135; A23K 10/18; A61K 45/06

(52) U.S. Cl. CPC A61K 35/74; A23L 33/135; A23V 2250/206; A61K 45/06; A23V 2002/00; A23K 10/18

(57) ABSTRACT

The present invention relates to a composition comprising a therapeutically effective amount of Bacteroides thetaiotaomicron or an extract thereof for use in the prevention or treatment of an infection in a subject who is immunodepressed, who is immunosuppressed, who has an immature immune system, and/or who has a weakness, and in which the composition comprises at most six species of bacteria and/or extracts thereof. The infections concerned are mainly intestinal infections, in particular those which affect non-human mammals and/or humans.

The present invention relates to the field of medicine and more specifically to the field of infections, in particular intestinal infections.

### BACKGROUND OF THE INVENTION

Infection refers to invasion by, and then multiplication of, microorganisms within an organ of a living body. These microorganisms may be viruses, bacteria, parasites or yeasts. The organism will activate defense processes to eradicate the undesirable microorganism.

Intestinal infections are infections that affect one or more segments of the intestine (small intestine, cecum, colon, rectum). These infections may be caused by a virus (such as rotavirus), a bacterium (such as Salmonella, Staphylococcus or Shigella), a parasite (such as amoebas, Giardia lamblia or worms) or a yeast (such as Candida albicans). The name given to the infection differs according to the part of the intestine affected: it is called enteritis when the small intestine is affected, colitis for the colon, sigmoiditis for the lower part of the colon, or proctitis for the rectum. These infections generally manifest as diarrhea and vomiting, which may be accompanied by nausea, stomach pain and fever. A principal complication of intestinal infections is dehydration, especially in the young and in the elderly. Some of these infections are particularly serious. One such example is cholera, a highly contagious and deadly acute intestinal infection which currently affects Central and Western Africa, or intestinal tuberculosis.

Globally, intestinal infections cause no fewer than 1.7 billion cases of diarrhea each year and are the second-leading cause of mortality in children under the age of 5 years (760,000 child deaths per year). In developing countries, children under the age of 3 years suffer an average of three diarrhea episodes per year, each of these episodes depriving such children of the nutritive elements essential to their growth. Thus, intestinal infections are also a major cause of child malnutrition (WHO; see the WHO website at the following link: http://www.who.int/mediacentre/factsheets/fs330/en/). Beyond these purely medical aspects, intestinal infections also have very high economic and social costs.

Controlling infections, in particular intestinal infections, is thus a major public health issue and there remains today a great need to develop new treatments for preventing or curing these infections.

Although mammals are born sterile (their digestive tract is microorganism-free), immediately after their birth their mucosa are colonized by a variety of microorganisms. Thus, colonization of the intestinal mucosal surface rapidly increases after birth to form an intestinal microflora containing roughly 10¹⁴ microorganisms. A mutually beneficial relationship is then established between the host and the microorganisms occupying its intestine. However, the intestinal mucosa is a delicately balanced environment, and it may indeed be invaded by pathogenic microorganisms that use this mucosa as a path into the organism and consequently cause infections. The role of the immune system is to activate differential responses to commensal (beneficial) and pathogenic microorganisms.

An adult's intestinal flora is thus a first line of defense against infection by providing, simply by its presence, resistance to any colonization by pathogenic microorganisms. The intestinal flora also takes part in food digestion and energy metabolism.

When antibiotic treatments are used against pathogens to control infection, the intestinal flora is partially destroyed because the antibiotics are generally not sufficiently selective. This destruction has the paradoxical effect of promoting the development of new intestinal infections. Furthermore, the development of resistance to antibiotics makes their use less and less effective.

It is thus urgent to propose and to develop novel methods for preventing and treating intestinal infections which will make it possible to effectively control the pathogenic microorganisms responsible for infections while preserving the integrity of the intestinal microflora.

### SUMMARY OF THE INVENTION

The objective of the present invention is to propose a novel composition for effectively controlling infections while preserving the intestinal microflora. The inventors discovered that administration of Bacteroides thetaiotaomicron (B. thetaiotaomicron) enables to mature the immune system and thus to combat infections. Furthermore, since B. thetaiotaomicron is one of the bacterial species naturally present in the intestinal microflora, its administration will not alter the microflora but will, on the contrary, have the effect of strengthening the defenses of the intestinal microflora against pathogenic bacteria.

Thus, the present invention concerns, in a first aspect, a composition comprising B. thetaiotaomicron and/or an extract thereof, for use in the prevention or treatment of infection in a subject who is immunodepressed, who is immunosuppressed, who has an immature immune system and/or who has a frailty, and wherein the composition comprises at most six bacterial species and/or extracts thereof.

In a particular embodiment, the composition comprises B. thetaiotaomicron and/or an extract thereof as the sole bacterium or bacterial extract.

Preferably, the subject has an infection.

Preferably, the infection according to the present invention is an intestinal infection, more preferably an infection selected from the group consisting of esophagitis, gastritis, enteritis, colitis, sigmoiditis, proctitis, peritonitis or a combination thereof.

The infection according to the present invention may be a bacterial, viral, parasitic or yeast infection, or a combination thereof.

In particular, the infection may be a bacterial infection, preferably selected from the group consisting of Vibrio cholerae, Vibrio parahaemolyticus, Escherichia coli, Shigella sonnei, Shigella flexneri, Shigella dysenteriae, Clostridium difficile, Clostridium perfringens, Clostridium botulinum, Salmonella enteritidis, Salmonella typhi, Salmonella paratyphi, Yersinia enterocolitica, Yersinia pseudotuberculosis, Campylobacter jejuni, Campylobacter coli, Campylobacter fetus, Helicobacter pylori, Listeria monocytogenes, Staphylococcus aureus, Bacillus cereus, Aeromonas, Mycobacterium tuberculosis or a combination thereof.

The infection may also be a viral infection, preferably selected from the group consisting of rotavirus, Norwalk virus, adenovirus, calicivirus, coronavirus, enterovirus, astrovirus or a combination thereof.

The infection may also be caused by a parasite, preferably selected from the group consisting of Entamoeba histolytica, Ascaris lumbricoides, Strongyloides stercoralis, Ancylostoma duodenale, Cyclospora cayetanensis, Cryptosporidium parvum, Microsporidium, Enterobius vermicularis, Giardia lamblia, Necator americanus, Taenia solium, Taenia saginata, Diphyllobothrium latum, Coccidia and Trichinella spiralis, or a combination thereof.

Finally, the infection may be caused by a yeast, preferably selected from the group consisting of Candida albicans, Candida parapsilosis, Candida tropicalis, Candida krusei, Candida guilliermondii, Torulopsis glabrata or combinations thereof.

The composition according to the invention may also be administered orally.

The subject of the invention is an animal, preferably a non-human mammal, more preferably a mammal selected from the group consisting of dogs, cats, horses, cows, pigs, sheep, goats, rabbits, hamsters, guinea pigs, hens, chickens and non-human primates. Particularly preferably, the subject of the invention is a newborn animal.

Alternatively, the subject of the invention is a human, preferably a human selected from the group consisting of a newborn, a child and an elderly person.

The composition according to the invention may also be a functional food or a dietary supplement.

The composition according to the invention may be administered in combination with a prebiotic, preferably selected from the group consisting of oligofructose, inulin, galacto-oligosaccharides, lactulose, human milk oligosaccharides, and derivatives or combinations thereof.

The composition according to the invention may also be administered in combination with an antibiotic, preferably selected from the group consisting of amoxicillin, ampicillin, vancomycin, neomycin sulfate, metronidazole, alatrofloxacin, tetracycline, moxifloxacin, azithromycin, bacampicillin, oxacillin, benzylpenicillin, clarithromycin, carbenicillin, cefadroxil, cephalexin, cefditoren, cefepime, cefmetazole, cefoperazone, cefprozil, cephalexin, ciprofloxacin, clarithromycin, clindamycin, daptomycin, dicloxacillin, erythromycin, fidaxomicin, gemifloxacin, sulfamethoxazole, kanamycin, levofloxacin, lincomycin, lomefloxacin, ofloxacin, meropenem, nafcillin, nalidixic acid, tobramycin, piperacillin, polymyxin, trimethoprim, rifampin, streptomycin, trovafloxacin, and derivatives or combinations thereof.

Finally, the composition according to the invention may further comprise at least one other bacterium and/or extract thereof, preferably a bacterium selected from the group consisting of Bacteroides ovatus, Bacteroides fragilis, Bacteroides intestinalis, Bacteroides vulgatus, Bacteroides caccae, Bacteroides massiliensis, Parabacteroides distasonis, Odoribacter splanchnicus, Faecalibacterium prausnitzii, Escherichia coli, Ruminococcus gnavus, Roseburia intestinalis, Fusobacterium necrogenes, Bifidobacterium infantis, Bifidobacterium animalis, Bifidobacterium breve, Bifidobacterium lactis, Bifidobacterium longum, Bifidobacterium bifidum, Lactobacillus rhamnosus, Lactobacillus helveticus, Lactobacillus casei, Lactobacillus bulgaricus, Lactobacillus acidophilus, Lactobacillus johnsonii, Lactobacillus plantarum, Lactobacillus reuteri, Lactobacillus salivarius, Streptococcus thermophilus, Bacillus clausii, Enterococcus faecium and combinations thereof.

### BRIEF DESCRIPTION OF THE DRAWINGS

FIG. 1. Microarray Study of Gene Expression in the Colon of Monocolonized Mice.

Gene expression in the colon of monocolonized (monoxenic), sterile (axenic group) and conventionalized mice was analyzed by microarray and represented using principal components analysis. The ellipses delimit the groups of mice studied: monocolonized (monoxenic group, in green), sterile (axenic group, in black) and conventionalized mice (conventional group, in red). The axes correspond to the principal components 1 (x axis) and 2 (y axis). The mice were monocolonized with Escherichia coli, Ruminococcus gnavus, Bacteroides thetaiotaomicron, Roseburia intestinalis, Saccharomyces boulardii or Candida albicans.

FIG. 2. Study of Expression of Key Genes of the Immune System in the Colon of Monocolonized Mice.

To confirm the microarray results, the genes of 8 immune system proteins were selected: T-bet, Gata3, IFNγ, TLR4, FoxP3, RORγt, IL-1β and CCL2. Expression of these genes was determined in the colon by RT-qPCR from their RNAs. The ΔΔCt method was used to quantify these genes using GAPDH as a reference gene and the group of sterile mice to calibrate the system. The results are expressed as means±SEM (n=5 per group). A statistical comparison was performed with the group of sterile mice using a Mann-Whitney test (*/**/***: p<0.05/0.01/0.001).

FIG. 3. Study of Immune System Activation in the Colon of Monocolonized Mice.

Colon samples were fixed with paraformaldehyde and then embedded in paraffin before being cut into four micrometer sections. These sections were then incubated for immunohistochemical analysis with rabbit polyclonal antibodies against CD3 (T cells, FIG. 3A), rat monoclonal antibodies against CD45R (B cells, FIG. 3B) and rat monoclonal antibodies against F4/80 (macrophages, FIG. 3C). Immunoglobulin A (IgA) production was determined by ELISA of feces samples (FIG. 3D). The results are expressed as means±SEM (n=5 per group). A statistical comparison was performed with the group of sterile mice using a Mann-Whitney test (*/**/***: p<0.05/0.01/0.001). Representative photographs were taken (×100 magnification).

### DETAILED DESCRIPTION OF THE INVENTION

Surprisingly, the inventors discovered that administration of B. thetaiotaomicron in the intestine of axenic mice, i.e., sterile mice (free of all microorganisms), leads to a maturation of the immune system of their host which is quite comparable to that observed in conventionalized mice, i.e., axenic mice to which a complete intestinal microflora has been transferred. In particular, the inventors noted infiltration of the intestinal mucosa of the host by T lymphocytes, B lymphocytes and macrophages but also maturation of the immunoglobulin A (IgA) system. This maturation of the immune system is essential for effective control of infections.

The bacterium B. thetaiotaomicron is one of the most abundant species of the phylum Bacteroidetes, in both humans and mice, Bacteroidetes being one of the three major phyla of the intestinal microflora (Qin J et al., 2010, Nature, 464, pp. 59-65). Being naturally present in the intestinal microflora, its administration will have no deleterious effect on the microflora but will, on the contrary, have the effect of strengthening the stimulation of the immune system and the defenses of the intestinal microflora against pathogenic bacteria and thus of better reinforcing action against infection or of preventing a new infection.

Thus, the present invention concerns, in a first aspect, a composition comprising B. thetaiotaomicron and/or an extract thereof, for use in the prevention or treatment of infection in a subject. Preferably, the subject is immunodepressed, is immunosuppressed, has an immature immune system and/or has a frailty. Preferably, said composition comprises at most six, five, four, three or two bacterial species and/or extracts thereof. In a particular embodiment, the composition comprises B. thetaiotaomicron and/or an extract thereof as the sole bacterium or bacterial extract.

The present invention also concerns the use of B. thetaiotaomicron or an extract thereof to prevent or treat infection in a subject, preferably a subject who is immunodepressed, who is immunosuppressed, who has an immature immune system and/or who has a frailty. Preferably, B. thetaiotaomicron or an extract thereof is used in combination with at most five, four, three, two or one bacterial species and/or extracts thereof. In a particular embodiment, B. thetaiotaomicron and/or an extract thereof is used as the sole bacterium or bacterial extract.

The present invention also concerns the use of Bacteroides thetaiotaomicron and/or an extract thereof for the preparation of a medicinal product intended for the prevention or treatment of infection in a subject, preferably a subject who is immunodepressed, who is immunosuppressed, who has an immature immune system and/or who has a frailty. Preferably, B. thetaiotaomicron or an extract thereof is used in combination with at most five, four, three, two or one bacterial species and/or extracts thereof. In a particular embodiment, B. thetaiotaomicron and/or an extract thereof is used as the sole bacterium or bacterial extract.

Finally, the present invention also concerns a method for preventing or treating infection in a subject, preferably a subject who is immunodepressed, who is immunosuppressed, who has an immature immune system and/or who has a frailty, comprising a step of administering Bacteroides thetaiotaomicron and/or an extract thereof to said subject. Preferably, B. thetaiotaomicron or an extract thereof is used in combination with at most five, four, three, two or one bacterial species and/or extracts thereof. In a particular embodiment, B. thetaiotaomicron and/or an extract thereof is used as the sole bacterium or bacterial extract.

The composition according to the invention comprises B. thetaiotaomicron and/or an extract thereof.

As used herein, the term “Bacteroides thetaiotaomicron” refers to any bacterial strain belonging to the species Bacteroides thetaiotaomicron, in particular strain VPI-5482 (ATCC 29149).

As used herein, the term “extract” or “cell extract” refers to any fraction obtained from the bacterial cell, notably a cell supernatant or a component thereof, cell debris, a cell wall or a component thereof, in particular a wall peptidoglycan, polysaccharide or protein, a DNA extract, the cytoplasm or a component thereof, a molecule or a molecular complex produced by the bacterium, enzymes or an enzyme preparation obtained from bacterial cells by chemical, physical and/or enzymatic treatment, and which is substantially without living cells. Preferably, when it is a matter of a B. thetaiotaomicron extract, this term refers to an extract capable of reproducing all or part of the effects of Bacteroides thetaiotaomicron.

The composition according to the invention may also comprise a mixture of Bacteroides thetaiotaomicron and Bacteroides thetaiotaomicron extracts, the addition of extracts strengthening the effect of Bacteroides thetaiotaomicron without increasing the number of bacteria administered to the subject.

The composition according to the invention comprises at most six bacterial species and/or extracts thereof. Thus, the composition according to the invention comprises Bacteroides thetaiotaomicron and/or an extract thereof and at most five other bacterial species and/or extracts thereof.

Preferably, the composition according to the invention comprises at most five bacterial species and/or extracts thereof. Alternatively, the composition according to the invention comprises at most four, at most three or at most two bacterial species and/or extracts thereof.

In an alternative embodiment, the composition according to the invention comprises exactly six, exactly five, exactly four, exactly three or exactly two bacterial species and/or extracts thereof.

In a particularly preferred embodiment, the composition according to the invention comprises a single bacterial species and/or extract thereof. In this case the species is B. thetaiotaomicron.

In another particular embodiment, said composition is administered in combination with another microorganism, microorganism extract or combinations thereof. Preferably, the bacterial species which may be present in the composition according to the invention in addition to B. thetaiotaomicron are compatible with the subject's intestinal microflora; more preferably they are commensal bacteria.

As used herein, the term “intestinal microflora” refers to all the microorganisms naturally present in the organs of the digestive tract. The terms “intestinal microflora,” “intestinal flora” and “intestinal microbiota” are equivalent in the context of the present invention and may be used interchangeably.

As used herein, the term “microorganism” refers to microscopic creatures such as bacteria, viruses, unicellular fungi (yeasts) and protists. The terms “microbe” and “microorganism” are equivalent and may be used interchangeably in the context of the present invention.

As used herein, the term “commensal microorganism,” for example commensal bacteria, refers to microorganisms naturally present in the subject's intestinal microflora and are generally beneficial to their host.

It is distinguished from a pathogenic microorganism, which generally has a harmful effect on its host. Pathogenic microorganisms are, in particular, responsible for infections.

Preferably, the bacterial species which may be present in the composition according to the invention in addition to B. thetaiotaomicron are selected from the group consisting of bacteria belonging to the phyla Bacteroidetes, Firmicutes, Actinobacteria, and Proteobacteria. More preferably, the bacterial species which may be present in the composition according to the invention in addition to B. thetaiotaomicron belong to a genus selected from the group consisting of Bacteroides, Parabacteroides, Clostridium, Fusobacterium, Eubacterium, Faecalibacterium, Ruminococcus, Peptococcus, Peptostreptococcus, Bifidobacterium, Escherichia, Lactobacillus, Odoribacter, Roseburia, Streptococcus, Bacillus and Enterococcus. Even more preferably, the bacterial species which may be present in the composition according to the invention in addition to B. thetaiotaomicron are selected from the group consisting of Bacteroides ovatus, Bacteroides fragilis, Bacteroides intestinalis, Bacteroides vulgatus, Bacteroides infantis, Bacteroides caccae, Bacteroides massiliensis, Bacteroides distasonis, Parabacteroides distasonis, Odoribacter splanchnicus, Faecalibacterium prausnitzii, Escherichia coli, Ruminococcus gnavus, Roseburia intestinalis, Fusobacterium necrogenes, Bifidobacterium infantis, Bifidobacterium animalis, Bifidobacterium breve, Bifidobacterium lactis, Bifidobacterium longum, Bifidobacterium bifidum, Lactobacillus rhamnosus, Lactobacillus helveticus, Lactobacillus casei, Lactobacillus bulgaricus, Lactobacillus acidophilus, Lactobacillus johnsonii, Lactobacillus plantarum, Lactobacillus reuteri, Lactobacillus salivarius, Streptococcus thermophilus, Streptococcus faecalis, Streptococcus bovis, Bacillus clausii, Enterococcus faecalis, Enterococcus faecium, Clostridium bifermentans, Clostridium clostridioforme, Clostridium symbiosum, Clostridium innocuum, Clostridium ramosum and combinations thereof. Particularly preferably, the bacterial species which may be present in the composition according to the invention in addition to B. thetaiotaomicron are selected from the group consisting of Bacteroides ovatus, Bacteroides fragilis, Bacteroides intestinalis, Bacteroides vulgatus, Bacteroides caccae, Bacteroides massiliensis, Parabacteroides distasonis, Odoribacter splanchnicus, Faecalibacterium prausnitzii, Escherichia coli, Ruminococcus gnavus, Roseburia intestinalis, Fusobacterium necrogenes, Bifidobacterium infantis, Bifidobacterium animalis, Bifidobacterium breve, Bifidobacterium lactis, Bifidobacterium longum, Bifidobacterium bifidum, Lactobacillus rhamnosus, Lactobacillus helveticus, Lactobacillus casei, Lactobacillus bulgaricus, Lactobacillus acidophilus, Lactobacillus johnsonii, Lactobacillus plantarum, Lactobacillus reuteri, Lactobacillus salivarius, Streptococcus thermophilus, Bacillus clausii, Enterococcus faecium and combinations thereof. Alternatively, the bacterial species which may be present in the composition according to the invention in addition to B. thetaiotaomicron are selected from the group consisting of Bacteroides intestinalis, Bacteroides massiliensis, Parabacteroides distasonis, Odoribacter splanchnicus, Faecalibacterium prausnitzii, Ruminococcus gnavus, Roseburia intestinalis, Bifidobacterium infantis, Bifidobacterium animalis, Bifidobacterium breve, Bifidobacterium lactis, Bifidobacterium longum, Bifidobacterium bifidum, Lactobacillus helveticus, Lactobacillus bulgaricus, Lactobacillus johnsonii, Lactobacillus plantarum, Lactobacillus reuteri, Lactobacillus salivarius, Streptococcus thermophilus, Bacillus clausii, Enterococcus faecium and combinations thereof.

The composition according to the invention may further comprise at least one other microorganism and/or microorganism extract. Said microorganism is preferably compatible with the subject's intestinal microflora; more preferably it is a commensal microorganism. Preferably, the commensal microorganism is a yeast; more preferably the commensal microorganism is a yeast selected from the group consisting of Saccharomyces boulardii, Saccharomyces cerevisiae, Candida albicans and/or extracts thereof. In a particular embodiment, the composition according to the invention consists essentially of B. thetaiotaomicron and/or an extract thereof.

As used herein, the term “consists essentially of” refers to a composition according to the invention comprising no other microorganisms, in particular no other commensal species. More particularly, said composition comprises no other bacterium and does not comprise yeast.

The composition according to the invention may also be administered in combination with another therapeutic agent, preferably selected from the group consisting of a prebiotic, an antibiotic or a combination thereof. The composition according to the invention and the additional therapeutic agent may be administered simultaneously or sequentially. When they are administered sequentially, the additional therapeutic agent may be administered before or after administration of the composition according to the invention.

In a particular embodiment, said composition is administered in combination with a prebiotic. As used herein, the term “prebiotic” refers to any molecule having a prebiotic effect, i.e., promoting the growth or the activity of intestinal microorganisms beneficial to the health of their host. Prebiotics are generally short-chain oligosaccharides or polysaccharides consisting of roughly 2 to 20 monosaccharide units. They escape digestion in the small intestine and are potential substrates for hydrolysis and fermentation by intestinal bacteria. The prebiotic employed in the invention may specifically target one species or genus of intestinal microorganism or several species or genera of microorganisms, allowing to modulate the total number or the relative proportion of one or more intestinal microorganisms. The prebiotics according to the invention may in particular be substrates for Bacteroides thetaiotaomicron. Preferably, the prebiotics according to the invention are selected from the group consisting of oligofructose, inulin, galacto-oligosaccharides, lactulose, human milk oligosaccharides, and derivatives or combinations thereof.

In another particular embodiment, said composition is administered in combination with an antibiotic. The antibiotic employed may specifically target one species or genus of intestinal microorganism or several species or genera of microorganisms, in order to modulate the total number or the relative proportion of one or more intestinal microorganisms. Preferably, the antibiotics according to the invention are selected from the group consisting of amoxicillin, ampicillin, vancomycin, neomycin sulfate, metronidazole, alatrofloxacin, tetracycline, moxifloxacin, azithromycin, bacampicillin, oxacillin, benzylpenicillin, clarithromycin, carbenicillin, cefadroxil, cephalexin, cefditoren, cefepime, cefmetazole, cefoperazone, cefprozil, cephalexin, ciprofloxacin, clarithromycin, clindamycin, daptomycin, dicloxacillin, erythromycin, fidaxomicin, gemifloxacin, sulfamethoxazole, kanamycin, levofloxacin, lincomycin, lomefloxacin, ofloxacin, meropenem, nafcillin, nalidixic acid, tobramycin, piperacillin, polymyxin, trimethoprim, rifampin, streptomycin, trovafloxacin, and derivatives or combinations thereof.

In another particular embodiment, the composition according to the invention consists of B. thetaiotaomicron and/or an extract thereof. In particular, the composition according to the invention comprises no other microorganisms and is not administered in combination with another therapeutic agent.

Preferably, B. thetaiotaomicron is present in a therapeutically effective amount.

As used herein, the term “therapeutically effective amount” refers to an amount of Bacteroides thetaiotaomicron and/or extracts thereof capable of preventing or delaying the appearance of infection, or of curing or reducing the effects of infection. It is obvious that the amount to be administered may be adapted by a person skilled in the art to the subject to be treated and to the nature of the infection, etc. In particular, the dosing and administration regimens depend on the nature, the stage of development and the severity of the infection to be treated, as well as the weight, the age and the general state of health of the subject to be treated, or on the judgment of the prescribing physician.

As used herein, the term “treatment” refers to any act directed at improving the medical status of a person with an infection. The treatment may be directed at both improving the patient's state—i.e., diminishing the infection or certain symptoms thereof—and eradicating the infection or certain symptoms thereof. The treatment may also have the effect of preventing or slowing the progression of infection.

As used herein, the term “prevention” refers to any act directed at preventing or delaying the appearance of infection. The terms “prevention” and “prophylactic treatment” are equivalent and may be used interchangeably in the context of the present invention. The prevention may be directed at both subjects predisposed to infection, preferably intestinal infection, and non-predisposed subjects.

In order to ensure effective prevention and/or treatment of infection, the composition according to the invention may be delivered to its site of action in the intestine in suppository form, in enema form, or orally, preferably orally. A person skilled in the art is capable of selecting any suitable formulation, vehicle, excipient, diluent or stabilizer. In particular, formulations of the composition include, but are not limited to, solid formulations such as tablets, pills and capsules; liquid formulations such as beverages and suspensions. Preferably, the composition is delivered orally and arrives at its site of action in the intestine without being damaged by the stomach's acidity, for example thanks to an enteric coating. Alternatively, the composition may be administered in the form of a functional food, a dietary supplement, a nutraceutical, a yogurt, a health bar, or other products of similar type.

The composition according to the invention may be administered in a single dose or in several doses according to the formulation, the subject, the subject's state of health, and the infection to be treated. When several doses of the composition are administered, they may be distributed over one or more days. Preferably, the composition according to the invention is administered in a single dose per day of administration. Alternatively, the subject receives several doses of the composition per day of treatment.

The composition according to the invention may be administered every day, every two days, once per week, once per month or once per year, preferably once per day. The periodicity of administration of the composition depends on many parameters, such as the global period the composition is administered, the subject's age and/or the seriousness of the infection to be prevented or treated. The periodicity of administration of the composition may also change over time, for a given subject, notably according to the progression of the subject's infection, the subject's risk of infection and/or the subject's general state of health.

The composition according to the invention may be administered for a maximum period of about 31 days, for a period of about 1 month to about 24 months, or for at least 2 years.

In a first embodiment, the composition according to the invention may be administered to the subject for a period of 1 day to about 31 days, preferably for a period of about 2 days to about 21 days, more preferably for a period of about 7 days to at about 14 days. An administration period of the composition of less than or equal to one month is particularly suited to acute treatment of infection or to prevention of an immediate risk of infection. Alternatively, the composition according to the invention may be administered throughout the period of infection or throughout the period of risk of infection.

In a second embodiment, the composition according to the invention may also be administered to the subject for a period of about 1 month to about 24 months, preferably for a period of about 2 months to about 12 months, more preferably for a period of about 3 months to about 6 months. An administration period of the composition of about 1 month to about 24 months is particularly suited to prevention of infection. Alternatively, the composition according to the invention may be administered throughout the period during which the subject has increased risk of infection.

Finally, in a third embodiment, the composition according to the invention may be administered to the subject for at least 2 years, preferably for at least 5 years, more preferably for at least 8 years. An administration period of the composition of at least 2 years is particularly suited to the prevention or treatment of chronic infection. Alternatively, the composition according to the invention may be administered throughout the life of the subject.

The composition according to the invention may be administered for the prevention or treatment of any infection, preferably an intestinal infection. An intestinal infection is an infection affecting an organ of the intestine, namely the esophagus, the stomach, the small intestine, the cecum, the colon or the rectum. The name given to the infection differs according to the organ of the intestine affected: it is called esophagitis when the esophagus is affected, gastritis when the stomach is affected, enteritis when the small intestine is affected, colitis for the colon, sigmoiditis for the lower part of the colon, or proctitis for the rectum. The intestinal infection according to the invention is preferably selected from the group consisting of esophagitis, gastritis, enteritis, colitis, sigmoiditis, proctitis, peritonitis, intestinal tuberculosis or a combination thereof.

An intestinal infection may be caused by various pathogenic microorganisms, such as viruses, bacteria, parasites, yeasts or combinations thereof. In a particular embodiment, the intestinal infection is caused by a virus, a parasite, a yeast or a combination thereof.

In a particular embodiment, the intestinal infection is caused by a virus, preferably a virus other than rotavirus.

In a preferred embodiment, the intestinal infection is caused by a virus selected from the group consisting of rotavirus, Norwalk virus, adenovirus, calicivirus, coronavirus, enterovirus, astrovirus or a combination thereof. In a more preferred embodiment, the intestinal infection is caused by a virus selected from the group consisting of Norwalk virus, adenovirus, calicivirus, coronavirus, enterovirus, astrovirus or a combination thereof.

In another particular embodiment, the intestinal infection is caused by a bacterium; preferably the intestinal infection is caused by a bacterium other than bacteria of the species Clostridium difficile, more preferably other than bacteria of the genus Clostridium. Alternatively, the intestinal infection is caused by a bacterium other than bacteria of the species Salmonella enteritidis, preferably other than bacteria of the genus Salmonella. In a preferred embodiment, the intestinal infection is caused by a bacterium other than bacteria of the species Salmonella enteritidis and Clostridium difficile. In a particularly preferred embodiment, the intestinal infection is caused by a bacterium other than bacteria of the genera Salmonella and Clostridium.

In another preferred embodiment, the intestinal infection is caused by a bacterium selected from the group consisting of Vibrio cholerae, Vibrio parahaemolyticus, Escherichia coli, Shigella sonnei, Shigella flexneri, Shigella dysenteriae, Clostridium difficile, Clostridium perfringens, Clostridium botulinum, Salmonella enteritidis, Salmonella typhi, Salmonella paratyphi, Yersinia enterocolitica, Yersinia pseudotuberculosis, Campylobacter jejuni, Campylobacter coli, Campylobacter fetus, Helicobacter pylori, Listeria monocytogenes, Staphylococcus aureus, Bacillus cereus, Aeromonas, Mycobacterium tuberculosis or a combination thereof.

More preferably, the intestinal infection is caused by a bacterium selected from the group consisting of Vibrio cholerae, Vibrio parahaemolyticus, Escherichia coli, Shigella sonnei, Shigella flexneri, Shigella dysenteriae, Clostridium perfringens, Clostridium botulinum, Salmonella typhi, Salmonella paratyphi, Yersinia enterocolitica, Yersinia pseudotuberculosis, Campylobacter jejuni, Campylobacter coli, Campylobacter fetus, Helicobacter pylori, Listeria monocytogenes, Staphylococcus aureus, Bacillus cereus, Aeromonas, Mycobacterium tuberculosis or a combination thereof.

Particularly preferably, the intestinal infection is caused by a bacterium selected from the group consisting of Vibrio cholerae, Vibrio parahaemolyticus, Escherichia coli, Shigella sonnei, Shigella flexneri, Shigella dysenteriae, Yersinia enterocolitica, Yersinia pseudotuberculosis, Campylobacter jejuni, Campylobacter coli, Campylobacter fetus, Helicobacter pylori, Listeria monocytogenes, Staphylococcus aureus, Bacillus cereus, Aeromonas, Mycobacterium tuberculosis or a combination thereof.

Alternatively, the intestinal infection is caused by a bacterium selected from the group consisting of Vibrio cholerae, Vibrio parahaemolyticus, Escherichia coli, Shigella sonnei, Shigella flexneri, Shigella dysenteriae, Clostridium difficile, Clostridium perfringens, Clostridium botulinum, Yersinia enterocolitica, Yersinia pseudotuberculosis, Campylobacter jejuni, Campylobacter coli, Campylobacter fetus, Helicobacter pylori, Listeria monocytogenes, Staphylococcus aureus, Bacillus cereus, Aeromonas, Mycobacterium tuberculosis or a combination thereof.

Alternatively, the intestinal infection may also be caused by a bacterium selected from the group consisting of Vibrio cholerae, Vibrio parahaemolyticus, Escherichia coli, Shigella sonnei, Shigella flexneri, Shigella dysenteriae, Salmonella enteritidis, Salmonella typhi, Salmonella paratyphi, Yersinia enterocolitica, Yersinia pseudotuberculosis, Campylobacter jejuni, Campylobacter coli, Campylobacter fetus, Helicobacter pylori, Listeria monocytogenes, Staphylococcus aureus, Bacillus cereus, Aeromonas, Mycobacterium tuberculosis or a combination thereof.

In another particular embodiment, the intestinal infection is caused by a parasite, preferably a parasite selected from the group consisting of Entamoeba histolytica, Ascaris lumbricoides, Strongyloides stercoralis, Ancylostoma duodenale, Cyclospora cayetanensis, Cryptosporidium parvum, Microsporidium, Enterobius vermicularis, Giardia lamblia, Necator americanus, Taenia solium, Taenia saginata, Diphyllobothrium latum, Coccidia and Trichinella spiralis or a combination thereof.

In still another particular embodiment, the intestinal infection is caused by a yeast, preferably a yeast selected from the group consisting of Candida albicans, Candida parapsilosis, Candida tropicalis, Candida krusei, Candida guilliermondii, Torulopsis glabrata or a combination thereof.

The composition according to the invention may be intended for pharmaceutical use or for veterinary use. As used herein, the term “subject” refers to an animal, preferably a mammal. The terms “subject” and “patient” are equivalent and may be used interchangeably in the context of the present invention.

The subject of the invention may be healthy or may have an infection. In a particular embodiment, the subject of the invention may have an infection; preferably, the subject of the invention has an intestinal infection. In an alternative embodiment, the subject of the invention does not have an infection; preferably, the subject does not have an intestinal infection. However, the subject may have an increased risk of infection.

The subject of the invention may be immunodepressed, may be immunosuppressed, may have an immature immune system, may have an abnormal intestinal microflora and/or may have a frailty.

In a preferred embodiment, the subject of the invention is immunodepressed, is immunosuppressed, has an immature immune system and/or has a frailty.

In a particularly preferred embodiment, the subject of the invention is immunodepressed, is immunosuppressed and/or has a frailty.

The subject of the invention may be immunodepressed. As used herein, the terms “immunodepressed” and “immunodeficient” are equivalent and may be used interchangeably. As used in the invention, the term “immunodepressed” refers to a state in which the subject has weakened immune defenses. Immunodepressed individuals are incapable of properly managing microorganisms that, under normal conditions, present no danger.

The subject of the invention may be immunosuppressed. As used herein, the term “immunosuppressed” refers to a state in which the subject no longer has immune defenses.

The subject of the invention may have an immature immune system. As used herein, the term “immature immune system” refers to a state in which the immune system has not yet reached maturity. The immune defenses of an immature immune system are less effective in controlling pathogens. In particular, newborns have an immature immune system, particularly preterm newborns. The more preterm the newborn, the less mature its immune system.

The subject of the invention may have a frailty. As used herein, the term “frailty” refers to a clinical syndrome reflecting a decreased reserve of physiological capacities that alters stress-adaptation mechanisms; its clinical expression is modulated by comorbidities and by psychological, social, economic and behavioral factors. The frailty syndrome is a marker of risk of mortality and of negative events, notably incapacities, falls, hospitalization and institutionalization. Age is a major determinant of frailty but on its own does not explain this syndrome. Treating the determinants of frailty may reduce or delay its consequences.

The subject of the invention may have an abnormal intestinal microflora. As used herein, the term “abnormal intestinal microflora” refers to an intestinal microflora unsuitable for effectively protecting the subject against intestinal infections. A subject may have an abnormal intestinal microflora following antibiotic treatment. This abnormal intestinal microflora may also be the consequence of a congenital abnormality.

The subject of the invention may be a non-human mammal or a human mammal.

In a particular embodiment, the subject of the invention is a human. The subject may thus be a newborn, a child, an adolescent, an adult or an elderly person. In a preferred embodiment, the subject is a human newborn or a child, more preferably a human newborn. In an alternative embodiment, the subject is an elderly person. As used herein, the term “newborn” refers to a human being under the age of 12 months, preferably under the age of 6 months, more preferably under the age of 3 months, and particularly preferably under the age of 1 month. In another preferred embodiment, the human subject is a child. As used in the present invention, the term “child” refers to a human being between the ages of 1 year and 12 years, preferably a human being between the ages of 1 year and 8 years, and more preferably a human being between the ages of 1 year and 5 years. In still another preferred embodiment, the human subject is an elderly person. As used in the present invention, the term “elderly person” refers to a human being aged 60 years or older, preferably a human being aged 65 years or older, and more preferably a human being aged 70 years or older.

In a particular embodiment, the subject of the invention is a preterm mammal, preferably human, more preferably a human newborn or a child, particularly preferably a human newborn.

As used herein, the term “preterm” refers to a subject born before term, i.e., before 40 weeks of gestation (WG), preferably before 37 WG. The preterm subject may be slightly preterm (from 35 to 37 WG), moderately preterm (from 32 to 34 WG), very preterm (from 27 to 31 WG), or extremely preterm (from 22 to 26 WG).

In the field of veterinary applications, the subject of the invention may be a non-human mammal, preferably a pet or a livestock animal, more preferably an animal selected from the group consisting of dogs, cats, horses, cows, pigs, sheep, goats, rabbits, hamsters, guinea pigs, hens, chickens and non-human primates. Particularly preferably, the subject of the invention is moreover a newborn. As used herein, the term “newborn” refers to an animal under the age of 12 months, preferably under the age of 6 months, more preferably under the age of 3 months, and particularly preferably under the age of 1 month. In particular, the subject of the invention may not be weaned.

In a second aspect, the present invention also relates to a kit for treating or preventing infection in a subject, preferably a subject who is immunodepressed, who is immunosuppressed, who has an immature immune system and/or who has a frailty, said kit comprising (i) a composition comprising a therapeutically effective amount of B. thetaiotaomicron and/or an extract thereof, and optionally (ii) a prebiotic, (iii) an antibiotic and/or (iv) another microorganism and/or extract thereof and also optionally (v) instructions detailing the safe use of such a kit. Preferably, the other microorganisms are at most five, four, three, two or one other species of microorganisms and/or extracts thereof, in particular bacteria. In a particular embodiment, the one or more additional microorganisms are commensal, more preferably one or more commensal yeasts or extracts thereof.

Preferably, the other microorganisms are at most five, four, three, two or one other species of microorganisms and/or extracts thereof, in particular bacteria. In a particular embodiment, the one or more additional microorganisms are commensal, more preferably one or more commensal yeasts or extracts thereof.

The embodiments described for the first aspect of this invention are also comprised in this second aspect.

In a third aspect, the present invention also concerns the use of a kit for treating or preventing infection in a subject, preferably a subject who is immunodepressed, who is immunosuppressed, who has an immature immune system and/or who has a frailty, said kit comprising (i) a composition comprising a therapeutically effective amount of B. thetaiotaomicron and/or an extract thereof, and optionally (ii) a prebiotic, (iii) an antibiotic and/or (iv) another microorganism and/or extract thereof, and also optionally (v) instructions detailing the safe use of such a kit. Preferably, the other microorganisms are at most five, four, three, two or one other species of microorganisms and/or extracts thereof, in particular bacteria. In a particular embodiment, the one or more additional microorganisms are commensal, more preferably one or more commensal yeasts or extracts thereof.

The embodiments described for the first aspect of this invention are also comprised in this third aspect.

Despite having different meanings, the terms “comprising,” “having,” “containing” and “consisting of” may be used interchangeably throughout the description of the invention.

In the context of the present description, all the molecules and cells may optionally be isolated and/or purified.

Other features and advantages of the invention will become apparent upon reading the following examples provided on a purely illustrative and non-limiting basis.

### Examples

The inventors studied the effect of four bacteria of the intestinal microbiota, Escherichia coli, Ruminococcus gnavus, Bacteroides thetaiotaomicron and Roseburia intestinalis, and two yeasts, Saccharomyces boulardii and Candida albicans, on the colic immune system using monoxenic mouse models. Monoxenic mice are mice whose intestine has been colonized by only one microorganism, in the present case a bacterium or a yeast.

### Materials and Methods

Microorganisms

Four bacteria and two yeasts were used in this study: Escherichia coli AIEC LF82, Ruminococcus gnavus ATCC 29149, Bacteroides thetaiotaomicron VPI-5482 (ATCC 29148), Roseburia intestinalis L1-82 (DSM 14610), Saccharomyces boulardii CNCM I-745 (syn. HANSEN CBS 5926, Biocodex Laboratories) and Candida albicans SC5314.

Mono-Associations

Female axenic (microorganism-free) C3H/HeN mice (ANAXEM facility, INRA, France) were raised in sterile incubators. Bacterial suspensions (10⁸-10⁹ CFU in 400 μL), yeast suspensions (10⁷-10⁸ CFU in 400 μL), feces of conventional mice, or a control medium were administered by intragastric gavage to 6-week-old mice (8 mice per group). After 6 weeks of implantation, the mice were euthanized and several samples were taken, namely specimens and contents of the jejunum, the ileum, the cecum and the colon.

Histology

Colon samples intended for histological study were fixed with 4% paraformaldehyde (Electron Microscopy Sciences) and then embedded in paraffin before being cut into micrometer sections. These sections were then incubated for histological and immunohistochemical analyses.

Quantification of Immunoglobulins

Feces were homogenized in PBS (1 mL for 100 mg) and centrifuged. All the supernatants were collected and then frozen at −80° C. until use. Quantification of immunoglobulin A was performed by ELISA, following the recommendations of the manufacturer (Mabtech).

Gene Expression

Total RNA was isolated from colon samples using the RNeasy Mini Kit (Qiagen), following the manufacturer's recommendations. The integrity of the RNA was checked on a Bioanalyzer 2100 using RNA 6000 Nano Chips (Agilent Technologies), and all the samples had an RNA integrity number greater than 8.

Transcription profile analysis was performed on mouse colon samples using SurePrint G3 Mouse GE 8×60K Microarrays (Design ID: 028005, Agilent Technologies). The microarray results obtained were submitted to GEO (accession number: GSE63299). Furthermore, expression of a selection of mouse genes was studied in mouse colon cells by RT-qPCR.

Analysis of Results and Statistics

All the results presented in graphical form are expressed as means or as means±SEM (n=3 to 8 per group) and a statistical comparison with a control condition was systematically performed with a Mann-Whitney test.

Results

Global Transcriptome Analysis

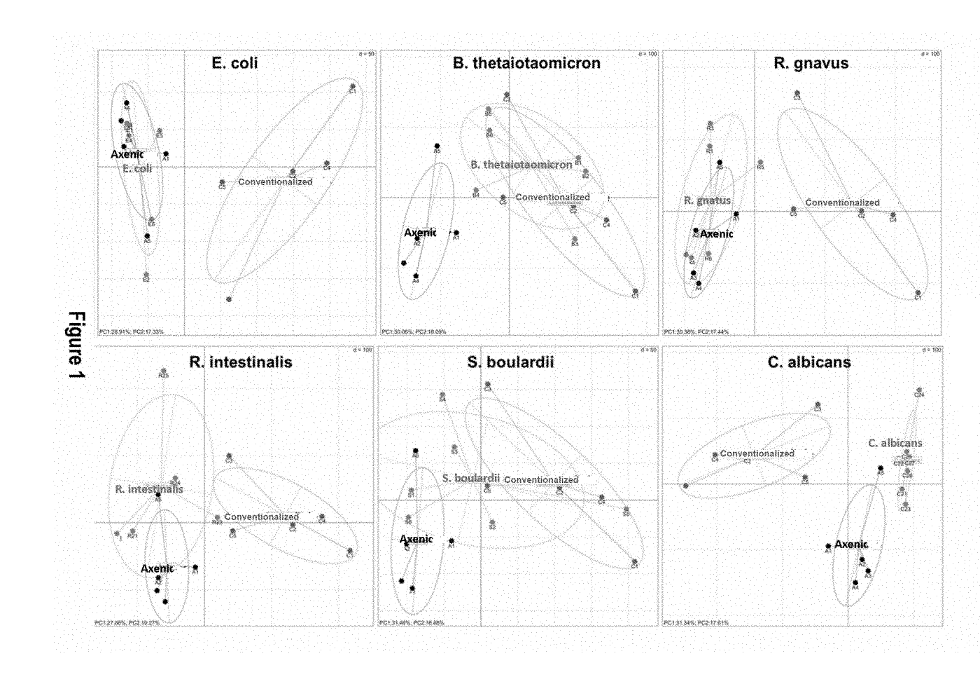

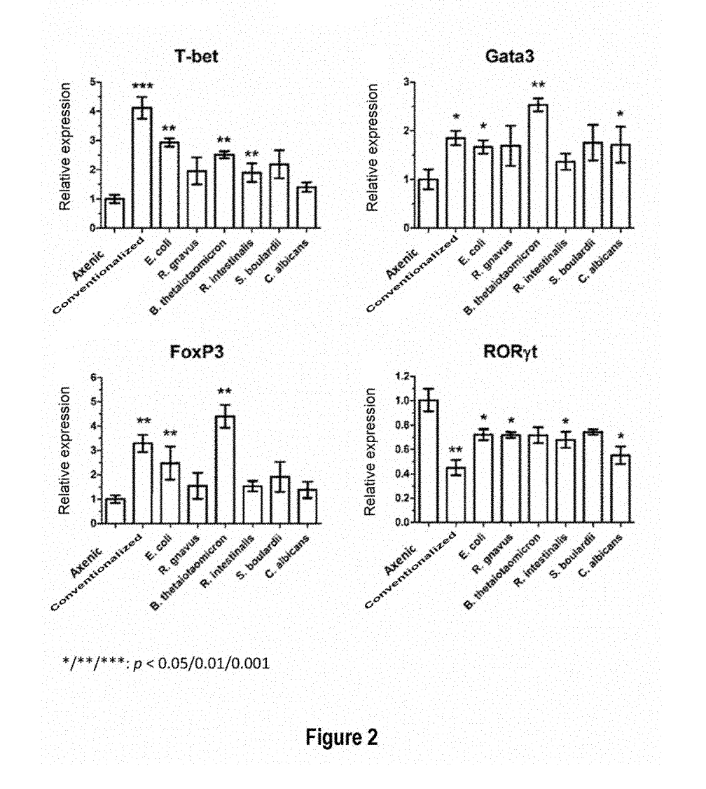

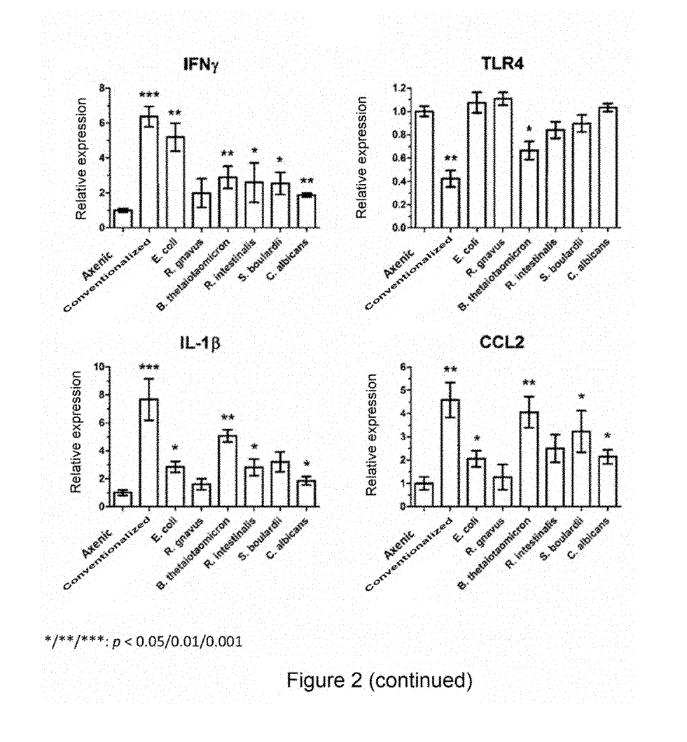

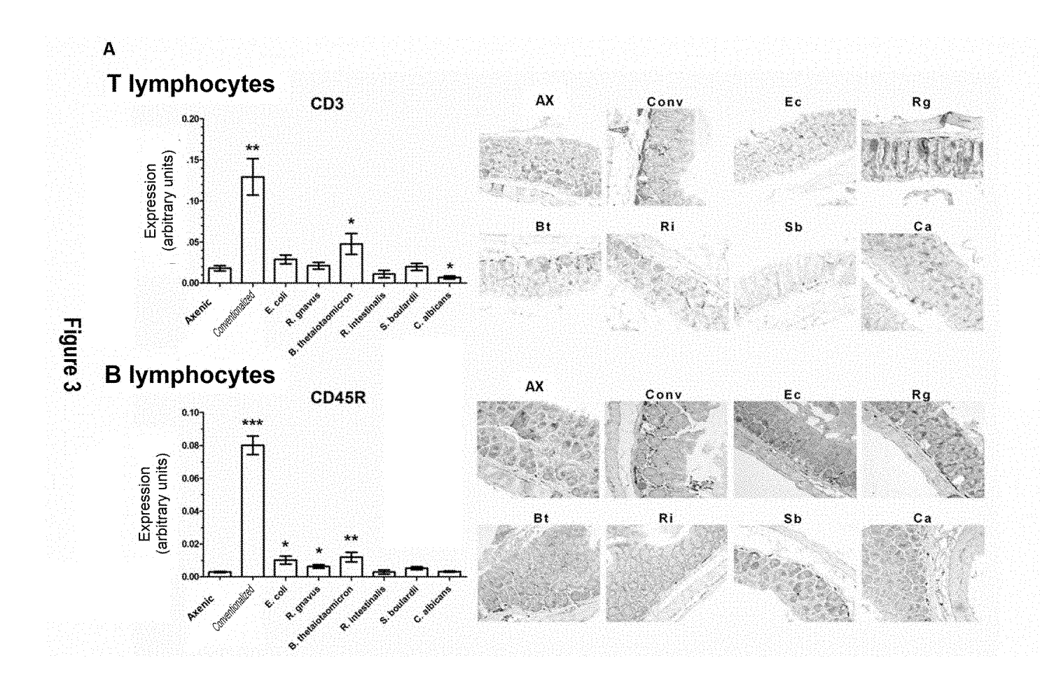

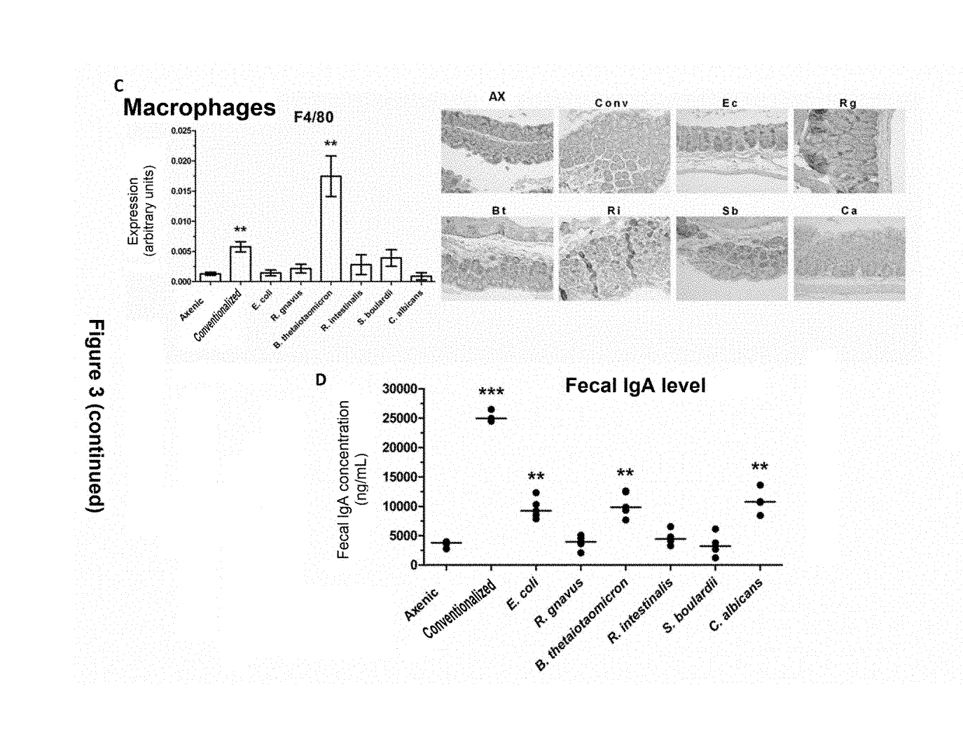

Analysis using a DNA-chip (microarray) technique was performed on the colon of monocolonized mice (6 groups corresponding to the 6 microorganisms of interest) and on that of axenic mice (no microorganisms) and of conventionalized mice (colonized with a normal whole microbiota). The transcriptome profile of the mice monocolonized with B. thetaiotaomicron, unlike those obtained with other microorganisms, is very close to that of the conventionalized mice (see FIG. 1).

Functional annotations of the microarray data, performed with the Cytoscape software, enabled to show that B. thetaiotaomicron particularly stimulates the genes involved in immune functions.

Stimulation of Expression of Genes Involved in Major Immune Functions by B. thetaiotaomicron

The inventors used qPCR to confirm the stimulation of expression of a selection of genes involved in major immune functions by B. thetaiotaomicron (see FIG. 2).

Analysis of Colonic Immune Populations and of Luminal Immunoglobulin A

By immunohistochemical analysis, the inventors showed that B. thetaiotaomicron induces infiltration of the colonic mucosa by various populations of immune cells: T lymphocytes, B lymphocytes and macrophages. This reflects a maturation of the immune system. Moreover, B. thetaiotaomicron also induces maturation of the IgA system in the intestinal mucosa (see FIG. 3). Although some aspects of maturation of the immune system are also observed with some of the other microorganisms studied, B. thetaiotaomicron is the only one to have a global impact, on all the parameters studied.

The inventors thus discovered that the bacterium B. thetaiotaomicron, on its own, was perfectly capable of maturing the intestinal immune system of its host.

1-16. (canceled)

17. A method of treating an infection in a subject with an immunodepressed, immunosuppressed or immature immune system, comprising administering to said subject a composition comprising B. thetaiotaomicron and/or an extract thereof said composition comprising at most six bacterial species and/or extracts thereof.

18. The method according to claim 17, wherein the subject has an infection.

19. The method according to claim 17, wherein said infection is an intestinal infection.

20. The method according to claim 17, wherein said infection is a bacterial, viral, parasitic or yeast infection or a combination thereof.

21. The method according to claim 20, wherein said infection is a bacterial infection.

22. The method according to claim 20, wherein said infection is a viral infection.

23. The method according to claim 20, wherein said infection is an infection caused by a parasite.

24. The method according to claim 20, wherein said infection is an infection caused by a yeast.

25. The method according to claim 17, wherein said composition is administered orally.

26. The method according to claim 17, wherein said subject is a non-human animal.

27. The method according to claim 26, wherein said animal is a newborn.

28. The method according to claim 17, wherein said subject is a human.

29. The method according to claim 17, wherein said composition is a functional food or a dietary supplement.

30. The method according to claim 17, wherein said composition is administered in combination with a prebiotic.

31. The method according to claim 17, wherein said composition is administered in combination with an antibiotic.

32. The method according to claim 17, wherein said composition further comprises at least one other bacterium and/or extract thereof, selected from the group consisting of Bacteroides ovatus, Bacteroides fragilis, Bacteroides intestinalis, Bacteroides vulgatus, Bacteroides caccae, Bacteroides massiliensis, Parabacteroides distasonis, Odoribacter splanchnicus, Faecalibacterium prausnitzii, Escherichia coli, Ruminococcus gnavus, Roseburia intestinalis, Fusobacterium necrogenes, Bifidobacterium infantis, Bifidobacterium animalis, Bifidobacterium breve, Bifidobacterium lactis, Bifidobacterium longum, Bifidobacterium bifidum, Lactobacillus rhamnosus, Lactobacillus helveticus, Lactobacillus casei, Lactobacillus bulgaricus, Lactobacillus acidophilus, Lactobacillus johnsonii, Lactobacillus plantarum, Lactobacillus reuteri, Lactobacillus salivarius, Streptococcus thermophilus, Bacillus clausii, Enterococcus faecium and combinations thereof.

33. The method according to claim 21, wherein the bacterial infection is an infection by a bacterium selected from the group consisting of Vibrio cholerae, Vibrio parahaemolyticus, Escherichia coli, Shigella sonnei, Shigella flexneri, Shigella dysenteriae, Clostridium difficile, Clostridium perfringens, Clostridium botulinum, Salmonella enteritidis, Salmonella typhi, Salmonella paratyphi, Yersinia enterocolitica, Yersinia pseudotuberculosis, Campylobacter jejuni, Campylobacter coli, Campylobacter fetus, Helicobacter pylori, Listeria monocytogenes, Staphylococcus aureus, Bacillus cereus, Aeromonas and Mycobacterium tuberculosis or a combination thereof.

34. The method according to claim 22, wherein the viral infection is an infection by a virus selected from the group consisting of rotavirus, Norwalk virus, adenovirus, calicivirus, coronavirus, enterovirus and astrovirus or a combination thereof.

35. The method according to claim 23, wherein the parasite is selected from the group consisting of Entamoeba histolytica, Ascaris lumbricoides, Strongyloides stercoralis, Ancylostoma duodenale, Cyclospora cayetanensis, Cryptosporidium parvum, Microsporidium, Enterobius vermicularis, Giardia lamblia, Necator americanus, Taenia solium, Taenia saginata, Diphyllobothrium latum, Coccidia and Trichinella spiralis or a combination thereof.

36. The method according to claim 24, wherein the yeast is selected from the group consisting of Candida albicans, Candida parapsilosis, Candida tropicalis, Candida krusei, Candida guilliermondii and Torulopsis glabrata or combinations thereof.
